(19) United States
(12) Patent Application Publication
(10) Pub. No.: US 2024/0397890 A1
(43) Pub. Date: Dec. 5, 2024

(54) ELECTROCHEMICAL LEACHING FOR NUTRIENT DELIVERY IN WATER

(71) Applicant: Battelle Memorial Institute, Richland, WA (US)

(72) Inventors: Chinmayee Venkata Subban, Seattle, WA (US); Scott J. Edmundson, Sequim, WA (US); Michael H. Huesemann, Sedona, AZ (US); Christopher R. Myers, Port Townsend, WA (US); Charles F. Hibbeln, Port Angeles, WA (US); Paul G. Marsh, Sequim, WA (US)

(21) Appl. No.: 18/680,826

(22) Filed: May 31, 2024

Related U.S. Application Data
(60) Provisional application No. 63/470,391, filed on Jun. 1, 2023.

(51) Int. Cl. A01G 33/00; C12M 1/36

(52) U.S. Cl. CPC A01G 33/00; C12M 41/48

(57) ABSTRACT

A method for enhancing photosynthetic productivity in aquatic environments through precision electrochemical nutrient delivery can involve deploying a pair of electrodes within a water-based matrix, where the first electrode functions as an anode and the second as a cathode, forming an electrochemical cell with the water serving as an electrolyte. At least one of these electrodes can be composed of or coated with an inorganic nutrient, facilitating a precise and low-concertation release of nutrients through controlled electrochemical nutrient delivery (CEND). A specified potential difference can be established between the electrodes to control the rate and amount of nutrient delivery. This such a potential difference can facilitate a precise addition of an inorganic nutrients into the water, such as to promote a growth or carbon capture capabilities of photoautotrophic organisms.

### CLAIM OF PRIORITY

This application claims the benefit of priority of Subban et al., U.S. Provisional Patent Application No. 63/470,391, titled “PRECISION ELECTROCHEMICAL FERTILIZATION FOR ENHANCED BIOLOGICAL PRODUCTIVITY,” filed on Jun. 1, 2023, which is incorporated by reference herein in its entirety.

### STATEMENT OF GOVERNMENT SUPPORT

This invention was made with Government support under Contract DE-AC0576RL01830 awarded by the U.S. Department of Energy. The Government has certain rights in the invention.

### BACKGROUND

Various environmental initiatives have been proposed for reducing or offsetting carbon dioxide (CO₂) emissions. Without being bound by theory, it is estimated that the ocean is capable of, in total, holding on the order of fifty times more inorganic carbon than the atmosphere does, and the oceans influence air-sea CO₂ exchange. Accordingly, the oceans can and do retain a substantial portion of anthropogenic CO₂ emissions. Augmentation of photosynthetic CO₂ uptake in marine environments could account for significant further global CO₂ capture and removal.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A is a diagram that illustrates an example of a technique for controlled electrochemical inorganic nutrient delivery to stimulate growth of or carbon capture via a photoautotroph population.

FIG. 1B shows a process of carbon fixation at an air-water interface via a photoautotroph.

FIG. 2 depicts an example of a system for nutrient delivery via controlled electrochemical nutrient delivery (CEND) in a marine environment.

FIG. 3A depicts an example of a system for nutrient delivery via controlled electrochemical nutrient delivery (CEND), including a dedicated anode and a dedicated cathode.

FIG. 3B depicts an example of a system for nutrient delivery via controlled electrochemical nutrient delivery (CEND), including a first switchable electrode and a second switchable electrode.

FIG. 4A is a chart showing an effect of controlled electrochemical nutrient delivery (CEND) on a Picochlroum celeri (P. celeri) biomass growth curve as optical density at 720 nm (OD720).

FIG. 4B is a chart showing an effect of controlled electrochemical nutrient delivery (CEND) on harvest densities in terms of ash free dry weight (AFDW).

FIG. 4C is a chart showing an effect of controlled electrochemical nutrient delivery (CEND) on harvest densities in terms of ash free dry weight (AFDW).

FIG. 4D is a chart showing an effect of controlled electrochemical nutrient delivery (CEND) on harvest densities in terms of ash free dry weight (AFDW).

FIG. 5A is a chart depicting a pulse time and a rest time of a multi-frequency program for controlled electrochemical nutrient delivery (CEND).

FIG. 5B is a chart depicting a relative effect of each stage of a multi-frequency program for controlled electrochemical nutrient delivery CEND.

FIG. 6A is a chart showing a controlled electrochemical dose delivery of iron (Fe), including an optical density (OD) change (680 nm) when supplied with varying total doses of electrochemically-delivered Fe.

FIG. 6B is a chart showing a controlled electrochemical dose delivery of iron (Fe), including a peak maximum photosynthetic growth rate at varying doses.

FIG. 6C is a chart showing a controlled electrochemical dose delivery of iron (Fe), including an amount of biomass extracted from a specified growth period at varying doses.

FIG. 7 is a flowchart describing a process for controlled electrochemical nutrient delivery (CEND) in water.

FIG. 8 illustrates generally a block diagram of a machine upon which any one or more of the techniques (e.g., methodologies) discussed herein may be performed.

### DETAILED DESCRIPTION

Stimulating photosynthetic activity in aquatic ecosystems can be of interest in certain fields, such as marine biology, environmental science, and hydroponics. Photosynthetic activity can be generally related to an availability of certain micronutrients in the water, which can enhance growth and metabolism of certain photosynthetic organisms such as algae, e.g., phytoplankton and seaweeds. An availability of iron, for instance, can influence growth of phytoplankton and other marine photosynthetic organisms. A natural distribution of these nutrients in certain marine environments can be uneven, such as resulting in large areas of nutrient-poor, yet otherwise habitable, marine environments known as oligotrophic waters.

Certain approaches to increase nutrient availability in certain marine environments can involve natural or anthropogenic processes. For example, natural events such as upwelling can involve transport of nutrients from relatively deep waters toward a surface, e.g., enhancing biological productivity. Anthropogenic approaches can involve artificial upwelling or artificially introducing chemical nutrients to stimulate biological activity, e.g., with an environmental goal of increasing carbon capture capabilities or resource productivity of certain marine environments. Such approaches can provide a countermeasure against rising atmospheric CO₂ levels.

However, such anthropogenic approaches can be challenging to control to avoid ecological impacts or to reverse or cease a process after it has been commenced, such as upon determining that an undesired effect is occurring. For example, certain approaches can involve a risk of stimulating algal blooms that can lead to hypoxic conditions, e.g., presenting a risk of affecting marine life adversely. For example, chemical fertilization, e.g., a direct addition of iron sulfate or a similar compound into ocean waters to stimulate phytoplankton growth, known as eutrophication, can involve a risk of subsequent depletion of oxygen levels in water. Further, such approaches can involve challenges related to the delivery of nutrients. For example, chemical fertilization can involve relatively large quantities of nutrients delivered in the open ocean lost to chemical transformations (e.g., via oxidation of iron in seawater) or physical dispersion before they can be metabolized by phytoplankton. The present inventors have recognized, among other things, a need for a system for controlled delivery of an inorganic nutrient, e.g., to a marine environment, such as for achieving a desired balance between enhancing photosynthetic productivity and maintaining the ecological integrity of an environment.

This document describes a technique for an enhancement of photosynthetic productivity in aquatic environments through nutrient supplementation. A technique for controlled electrochemical nutrient delivery (CEND) can involve establishing an electrochemical cell composed of electrodes placed within a water-based matrix, such as an ocean or aquaculture environment. The electrodes can be formed from or coated with materials including desired inorganic nutrients, such as one or more of iron (Fe), manganese (Mn), or zinc (Zn). Such inorganic nutrients can be leached via the CEND in a precise manner into the water-based matrix. For example, the CEND can involve controlled dispersal of the inorganic nutrients at a specified rate, dose, or concentration, such as to help promote release of the nutrients in the water in bioavailable forms and in precise quantities that can be specified based on a biological demand, e.g., of local aquatic organisms. Such a controlled release can promote efficient nutrient uptake photosynthetic organisms in the water, and also can avoid or mitigate certain ecological risks associated with other fertilization methods (e.g., direct dumping of large quantities of chemicals). In an example, the technique can facilitate real-time adjustment to a delivery rate of the inorganic nutrients or adjustment to a total quantity or concentration of the nutrients, such as based on feedback from environmental sensors or specified delivery profiles based on growth conditions or stages of biomass development.

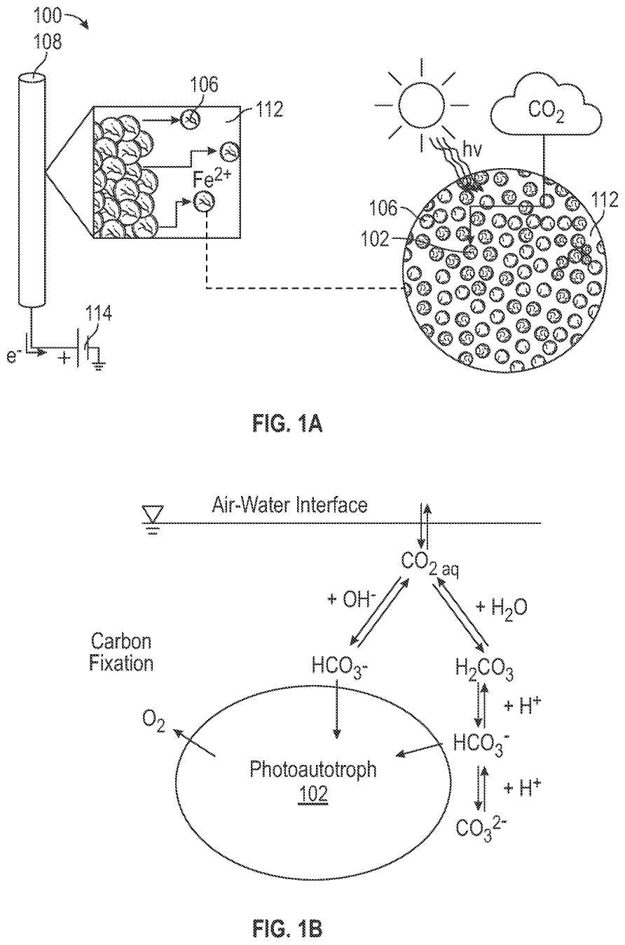

FIG. 1A is a diagram that illustrates an example of a technique for controlled electrochemical delivery of an inorganic nutrient to stimulation growth of or carbon capture via a photoautotroph population. The technique can be performed, e.g., using a system 100 for controlled electrochemical nutrient delivery (CEND) of an inorganic nutrient 106 via an electrode 108. The CEND can be powered by a low-voltage, e.g., renewable energy source, such as to promote a sustainability of the operation. This technique can be particularly advantageous in remote or open ocean settings where certain alternative nutrient delivery techniques are logistically challenging, not sustainable, or costly.

In an example a first electrode 108 can be placed within a water-based matrix 112. The first electrode can act as an anode and can establish an electrochemical cell with a second electrode as a cathode and the water-based matrix 112 as an electrolyte. The first electrode can be clad with the inorganic nutrient 106, which can include a metallic species such iron (Fe), zinc (Zn), manganese (Mn), cobalt (Co), copper (Cu), nickel (Ni), or other species capable of providing essential macronutrients or micronutrients to species of photoautotrophs. For example, such nutrients can be part of an alloy, part of a composite electrode, or coating on the electrode, facilitating controlled delivery of one or more type of inorganic nutrient 106 into the surrounding water upon the first electrode 108 being electrically excited.

In an example, the first electrode 108 and the second electrode can be excited to establish a specified potential difference using an energy source 114. In an example, the energy source 114 can include or be entirely consisting of renewable energy, such as solar, wind, or kinetic energy collected at or near the water-based matrix. The specified potential difference can be static or time-varying, such as maintained for relatively long periods of time (e.g., for hours) at a low voltage, such as less than five volts per cell pair, promoting energy efficiency and avoiding or mitigating undesired chemical reactions. The technique can also include a monitoring indicator of growth for photoautotrophs 102 in the matrix 112, such as for adjusting at least one parameter of the delivery of the inorganic nutrient 106 in response to the monitoring indicator. For example, such a response can involve modulating the specified potential difference and controlling the cumulative delivery of the inorganic nutrient 106 adjusting, e.g., a duration over which a potential is applied to the first electrode 108 and the second electrode. The monitoring indicator of growth may include one or more sensors or agents for detecting concentrations of respective growth constituents (e.g., ammonium, phosphate, iron, zinc), gas sensors for detecting dissolved O₂ and CO₂ levels for photosynthesis, temperature, pH, dissolved oxygen, or the like. Other types of sensors such as visual, chemical, or biochemical indicators can also be employed as appropriate for monitoring a particular type of photoautotroph population.

In an example, the specified potential can facilitate changing an oxidation state of a metallic species included in the first electrode 108 to elicit the electrochemical delivery of the metallic species. For example, where the metallic species comprises iron (Fe), and the oxidation state can be driven by the specified potential toward a state of Fe⁺² or Fe⁺³, or both. In certain examples, the specified potential may be selected such as to establish a ratio of oxidized species to reduced species in the range of about 0 to about 100 percent, such as in a range of about 20 to about 80 percent, a range of about 30 to about 70 percent, or another range.

FIG. 1B shows an example of a biochemical process of carbon fixation at an air-water interface via a photoautotroph. In an example, the technique described in FIG. 1A can be configured for use in certain aquatic environments, e.g., open or coastal marine settings and or alternatively in a controlled environment such as a hydroponic system or a raceway ponds. For example, upon electrical excitation within a water-based matrix 112 that includes dissolved carbon (e.g., atmospheric CO₂), a delivered nutrient 106 to the photoautotroph 102 can result in an uptake of carbon.

Photosynthetic marine activity can facilitate capture of atmospheric carbon as biomass. Certain essential nutrients can be limited in certain environments for photoautotroph 102 populations (e.g., phytoplankton, microalgae, cyanobacteria, seaweeds, seagrasses, mangroves, etc.) which can constrain a capacity for such carbon capture and fixation. For example, in a presence of natural light (or full-spectrum, artificial illumination) with sufficient essential nutrients, pigment molecules or other chemical components associated with the photosynthetic reaction center of the photoautotroph 102 can capture electromagnetic photosynthetically active radiation (PAR) as energy. Such energy can be used to drive ATP synthesis or other chemical reactions needed for biosynthesis and cellular maintenance. Such chemical reactions can involve an intake of bicarbonate (HCO₃⁻) or dissolved CO₂ from the water-based matrix 112, which can in turn facilitate conversion of inorganic carbon (e.g., CO₂ or HCO₃⁻) at or near an air-water interface (between an ambient atmosphere and the matrix 112) toward carbonate ion (CO₃²⁻), with oxygen (O₂) released as a product.

In an example, controlled electrochemical nutrient delivery (CEND) within the water-based matrix 112 as described in FIG. 1A can facilitate an increase in harvest densities of algae cultures by greater than 200%, such as greater than 300%, greater than 400%, greater than 500%, greater than 600%, greater than 700%, greater than 800%, or greater than 900% (e.g., when compared to a control or a non-electrochemically stimulated environment). For example, an electrically iron-dosed photoautotroph 102 population can capture CO₂ with at about 1.04±0.29 biomass productivity (grams per liter per day or g L⁻¹ day⁻¹), which can be about ten times more than certain iron-limited populations of a similar photoautotroph.

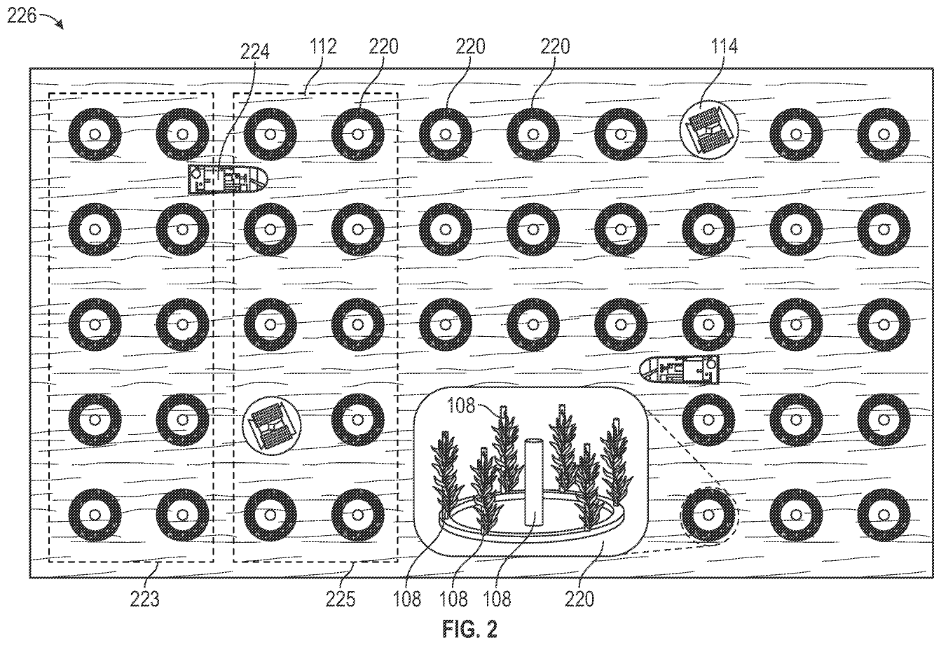

FIG. 2 shows an overhead view of an example of a system for nutrient delivery via controlled electrochemical nutrient delivery (CEND) in a marine environment. The system 200 can include an array of electrode configurations 220 positioned in a water-based matrix 112, such as powered using one or more energy sources 114. The array of electrode configurations 220, the one or more energy sources 114, or both, can be placed, propelled by, or otherwise recovered via a vessel 224, e.g., a boat, a ship, a barge, etc. In an example, the vessel 224 can be used, e.g., to harvest the photoautotroph 102 upon growth facilitated via the CEND. Here, the water-based matrix 112 can be an open or coastal marine environment including an aquatic reservoir, or other body of water such as an ocean, lake, sea, bay, lagoon, etc. The water-based matrix 112 can also be defined by a growth cell isolated from other growth cells, or a hydroponic reservoir.

An individual electrode configuration 220 can include a first electrode 108 and a second electrode 109. In an example, the first electrode 108 can act as a cathode (e.g., shaped and arranged as a ring electrode) and the second electrode 109 (e.g., shaped and arranged as a linear electrode and disposed within the ring electrode) can act as an anode, e.g., to comprise an electrochemical cell with the water-based matrix as an electrolyte. Alternatively or additionally, the second electrode can be shaped and arranged as a rod or a plate. At least one of the first and second electrodes 108 & 109 can be composed of or coated with inorganic nutrients, such as iron, zinc, or manganese. The individual electrode configuration 220 can also include or be electrically coupled to an excitation source. For example, the excitation source can use energy from the energy source 114 to establish or adjust a specified potential difference between the first and second electrodes, such as to facilitate controlled electrochemical nutrient delivery (CEND) of the desired inorganic nutrients. An individual electrode configuration 220 can include or use control circuitry configured to modulate the specified potential difference between a respective first electrode 109 and a respective second electrode 109, such as to control a cumulative inorganic nutrient delivery in the matrix 112. For example, the control circuitry, or another processor, can control electrode voltage, current, pulse-width, pulse-duration, pulse-frequency, or combinations thereof, to establish desired controlled electrochemical nutrient delivery (CEND) in a specified region of the matrix, or across a specified parametric timeline. The processor, control circuitry, or energy source 114 can also process data from sensor unit, such as a conductivity sensor, galvanic potential sensor, optical nutrient sensor, or combinations thereof.

In an example, the array of electrode configurations 220 can be arranged to establish a plurality of differentiated fields 223 and 225, e.g., with each of the differentiated fields associated with or selected based on one or more characteristic geological and ecological zones of the marine environment, or properties of the electrode materials used. The array of electrode configurations 220 can also be configured to enable dynamic electrode actuation. For example, excitation potentials can be applied to electrodes corresponding to particular locations in or about the array, to achieve directed generation and delivery of inorganic nutrients to the water-based matrix 112 in a spatially-localized or controllable manner. The array can also be configured for automated operation on an ongoing basis, such as to maintain a specified nutrient parameter within the matrix 112 or based on the one or more characteristics associated with geological or ecological zones in which the array is operated.

In an example, electrode configurations 220 can provide the CEND using energy from the one or more energy sources 114. The first and second electrodes can be excited using a source of renewable energy, such as solar energy, wind energy, wave energy, tide energy, ocean thermal energy conversion (OTEC), or heat-based energy sources. A source of renewable energy can generate an electrical output, either directly or using conversion from thermal or mechanical energy such as by capturing pneumatic force, hydraulic force, or combinations thereof. The specified potential difference can be generated using circuitry configured, for example, to produce a desired voltage variation between the first electrode 108 and the second electrode 109. For non-limiting examples, the specified potential difference can be at least 0.1V, 0.25V, 0.5V, 0.75V, 1V, 1.5V, 2V, 3V, 4V, 5V, or 6V. In some examples, the specified potential difference can be at least 1e-3V, 1e-2V, 1e-1V 0.1V, or less, such as to achieve a specified electrochemical nutrient delivery regime. At least one of the first and second electrodes 108 or 109 can also be impedance-adjusted, load-matched, or dynamically swapped or interchanged in an example to achieve the specified potential difference.

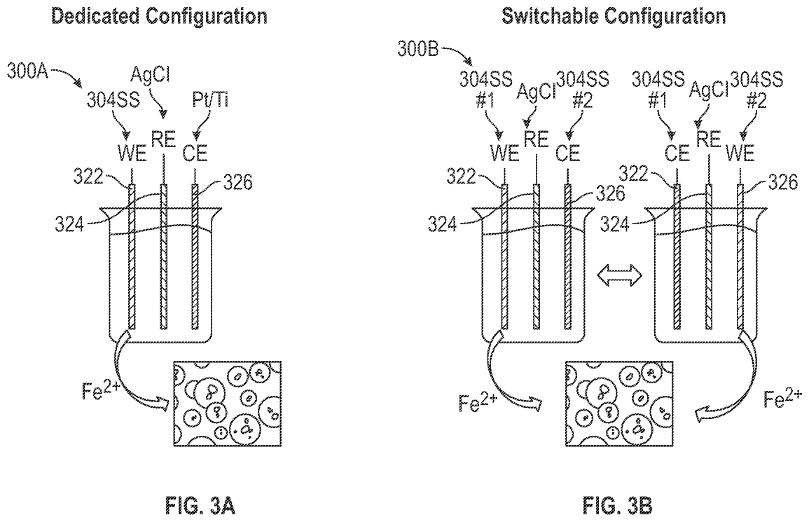

FIG. 3A depicts respective examples of a system for nutrient delivery via controlled electrochemical nutrient delivery (CEND), including a dedicated anode and a dedicated cathode. The system 300A can include at least one electrode configuration 220 disposed within the water-based matrix 112. The at least one electrode configuration 220 can include a working electrode (WE) 322, a reference electrode (RE) 324, and a counter electrode (CE) 326. Any of the WE 322, RE 334, and CE 326 can be similar to the first electrode 108 and the second electrode 109 as described with respect to FIG. 1A and FIG. 2. For example, the first electrode 108 or the second electrode 109 can be assigned as the WE 322, the RE 324, or the CE 326. As depicted in FIG. 3A, the electrode configuration 220 can include a dedicated anode and a dedicated cathode. Herein, “dedicated” means that a particular electrode is constructed and driven to function as only an anode or only a cathode and is not driven or physically configured to function as both an anode and a cathode. Generally, a voltage source can be used to control the rate of nutrient release from the anode and cathode materials, which can then control the number of electrochemical signals and nutrient dosing into the water-based matrix 112. In an example, the WE 322 can be formed of stainless steel, such as 304SS or 316SS, or any other alloy, metal, composite, or raw mineral-based electrode. The RE 324 can be formed of a compatible composition such as Ag/AgCl or Hg/Hg₂Cl₂ electrically connected to an electrical circuit. CE 326 can comprise another material such as platinum, titanium, copper, graphite, silver, or a combination thereof.

FIG. 3B depicts an example of a system for nutrient delivery via controlled electrochemical nutrient delivery (CEND), including a first switchable electrode and a second switchable electrode. The system 300B can include at least one electrode configuration 220 disposed within the water-based matrix 112. The at least one electrode configuration 220 can include a first composition electrode 332 and a second composition electrode 336, capable of switching between an anode configuration and a cathode configuration. Herein, “switching between an anode configuration and a cathode configuration” can mean the electrode can be excited by electrical pulses or other electrical means to selectively serve as a cathode for one duration, and be connected to serve as an anode for another duration. The characteristics of cathodic pulse operation can be set by amplitude, frequency, polarity, and other attributes. Such switching can facilitate using a voltage source to control the rate of nutrient delivery from the anode and cathode materials, and thereby create controlled timing of electrochemical signals and release of nutrients into the water-based matrix 112. Additionally or alternatively, the characteristics used to establish anodic pulsed operation can similarly affect nutrient delivery duration. In an example, the WE 322 can serve as an anode during a first duration of time and is then reversibly switched to cathodic operation by applying a different pulsed voltage or other electrical means. Concurrently, the CE 326 can serve as a cathode during the first period of time and is then reversibly switched to anodic operation by applying the different pulsed voltage or other electrical means. In the system 300B, the CE 326 can be formed of an alloy such as stainless steel 304SS or 316SS, or otherwise formed of the same material as the WE 322. In an example, the system 300B can include a plurality of electrode configurations 220, and respective electrodes WE 322 and CE 326 can be excited by modulated electrical excitation (e.g., polarity switched under control of a system controller or other processor) such as to perform precise CEND in a spatially-controllable manner within or nearby the water-based matrix 112.

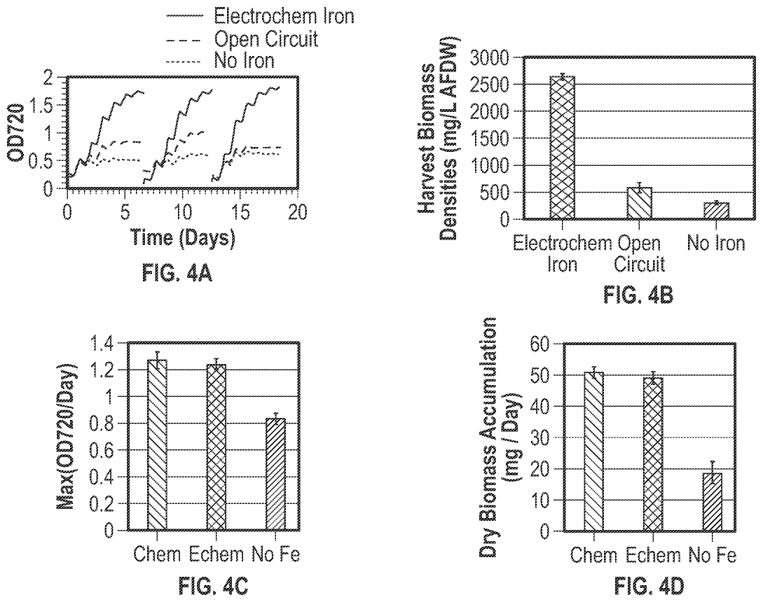

FIG. 4A is a chart showing an effect of controlled electrochemical nutrient delivery (CEND) on a P. celeri biomass growth curve as optical density at 720 nm (OD720). FIG. 4B is a chart showing an effect of CEND on harvest densities in terms of ash free dry weight (AFDW). In an example, after a plurality of days subject to CEND at a specified potential (e.g., about 6 days), P. celeri cultures can contained about 890% more biomass (or about nine times) as compared to other, iron-deficient P. celeri control cultures (e.g., about 2640±60 milligrams per liter (mg/L) vs. About 290±30 mg/L as ash free dry weight). For example, electrodes with no voltage applied (e.g., including only unstimulated Fe delivery via corrosion) can increase an algal harvest biomass by about 200% (about 580±90 mg/L compared to the no Fe control).

FIG. 4C is a chart showing an effect of controlled electrochemical nutrient delivery (CEND) on harvest densities in terms of ash free dry weight (AFDW). As demonstrated in FIG. 4C, an CEND technique for controlling delivery of iron (Fe) in a water-based matrix can result in comparable performance (e.g., exhibiting similar optical density (OD) based peak growth rates of a photoautotroph) as compared to chemical fertilization techniques for Fe delivery. This plot also shows a control of no supplementary Fe delivered to the water-based matrix by way of comparison.

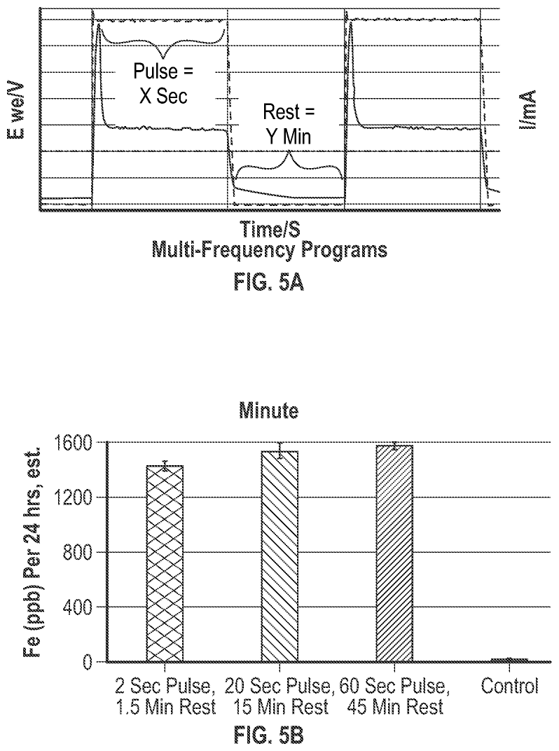

FIG. 5D is a chart showing an effect of controlled electrochemical nutrient delivery (CEND) on harvest densities in terms of ash free dry weight (AFDW). As demonstrated in FIG. 5D, a CEND technique for controlling delivery of iron (Fe) in a water-based matrix can result in comparable performance (e.g., exhibiting similar Biomass accumulation rates as normalized to extraction time) as compared to chemical fertilization techniques for Fe delivery. This plot also shows a control of no supplementary Fe delivered to the water-based matrix by way of comparison.

FIG. 5A is a chart depicting a pulse duration and a rest duration of a multi-frequency program for controlled electrochemical nutrient delivery (CEND). In an example, first and second electrodes of an electrode configuration (e.g., configuration 220 of FIG. 2, FIG. 3A, or FIG. 3B) can be excited via an excitation source (e.g., a controller, a microprocessor, a multiplexer, a source of energy, or a combination thereof) to establish a specified potential difference across the electrodes in a water-based matrix, e.g., establishing a time-varying potential. The time-varying potential can be controlled at a specified pulse repetition frequency (PRF) and a specified pulse width to define a program for eliciting delivery of the inorganic nutrient via the CEND.

FIG. 5B is a chart depicting a relative effect of each stage of a multi-frequency program for controlled electrochemical nutrient delivery (CEND). As shown in FIG. 5B, an electrode can be formed of an alloy, an intermetallic, or a metal-containing composite configured to deliver specified amount of the inorganic nutrient from the first electrode during the CEND. For example, the electrode can be formed of stainless-steel. In an example, a total amount of Fe delivered-via the stainless-steel electrode can be quantified (e.g., by mass percentage), e.g., using Inductively Coupled Plasma Optical Emission Spectrometry (ICP-OES) or a similar spectroscopy technique.

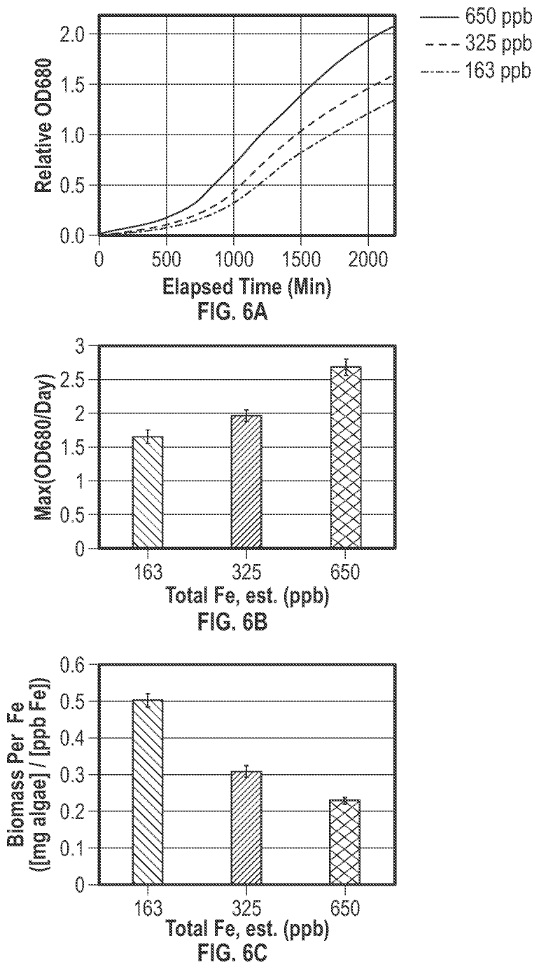

FIG. 6A is a chart showing a variable dose delivery of iron (Fe) via controlled electrochemical nutrient delivery (CEND), including an optical density (OD) change (680 nm) corresponding to photosynthetic enhancement when supplied with varying total doses of electrochemically delivered Fe.

FIG. 6B is a chart showing a dose delivery of iron (Fe) via controlled electrochemical nutrient delivery (CEND), including a maximum growth rate at varying doses. Increasing maximum growth rates can be correlated with an increase of a total Fe dose. In the depicted range of dosages, the effect can be approximately linear, resulting in about +0.002 optical density per day (OD/day) per parts per billion (ppb) Fc.

FIG. 6C is a chart showing a dose delivery of iron (Fe) via controlled electrochemical nutrient delivery (CEND), including an amount of biomass extracted from a specified growth period at varying total Fe doses. For example, the specified growth period can be about (72 hours). As shown in FIG. 6C, relatively smaller doses can yield increased efficiency in simulating algal biomass yield, e.g., with a biomass decreasing in a e.g., a non-linear fashion toward progressively higher total Fe dose.

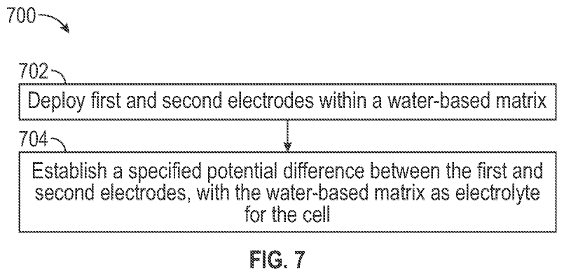

FIG. 7 is a flowchart describing a process for controlled electrochemical nutrient delivery (CEND) in water. For example, the process can be performed using the system 100, the system 200, the system 300A, or the system 300AB as depicted in FIG. 1A, FIG. 2, FIG. 3A, or FIG. 3B.

At 702, first and second electrodes (e.g., arranged in an electrode configuration such as configuration 220 of FIG. 2) can be deployed within a water-based matrix, e.g., via a vessel such as a boat, a ship, a raft, a barge, buoy, etc. and held in-place using moorings. The first electrode can be designated as the anode, and the second electrode can be designated as the cathode. In an example, the first electrode can be clad with or otherwise contain an inorganic nutrient (e.g., iron (Fc), manganese (Mn) or zinc (Zn).

In 704, a specified potential difference can be established between the first and second electrodes, such that the first electrode defines an anode in an electrochemical cell, and the second electrode defines a cathode in the electrochemical cell, with the water-based matrix providing an electrolyte for the cell. Such a potential difference can facilitate electrochemical reactions for a delivery of the inorganic nutrient into the water-based matrix. The potential difference can be maintained at less than five volts per cell pair, e.g., to optimize energy efficiency and prevent excessive electrolysis that could lead to undesired byproducts. Control of the potential difference via the first and second electrode can control a release of the inorganic nutrient from the first electrode into the water-based matrix. In an example, the oxidation state of the metallic species, such as iron, can be manipulated via the potential to enhance bioavailability, with the potential driven towards states such as Fe⁺² over Fe⁺³.

Throughout the nutrient delivery process, a growth and health of the photoautotrophs in the water-based matrix can be monitored. Accordingly, the potential difference or the duration of the applied potential can be adjusted, based on the monitoring, to promote nutrient delivery based on acquired data. For example, this can involve modulating the potential to an opposite polarity or adjusting the pulse width and frequency of the applied potential. Once a desired level of nutrient delivery is achieved, or if the system needs to be halted for any reason, the process can be terminated by suppressing the potential difference. The electrodes can then be retrieved out of the water matrix or left in place for future nutrient delivery cycles. The system can be powered by renewable energy sources, such as solar or wind energy, enhancing the sustainability of the method.

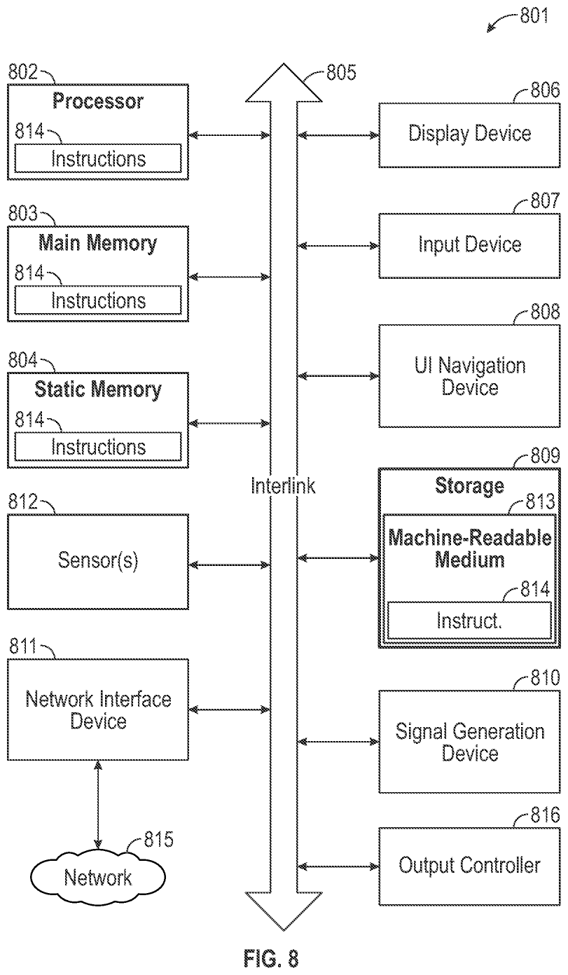

FIG. 8 illustrates generally an example of a block diagram of a machine 801 upon which any one or more of the techniques (e.g., methodologies) discussed herein may perform in accordance with some examples. In alternative embodiments, the machine 801 may operate as a standalone device or may be connected (e.g., networked) to other machines. In a networked deployment, the machine 801 may operate in the capacity of a server machine, a client machine, or both in server-client network environments. In an example, the machine 801 may act as a peer machine in peer-to-peer (P2P) (or other distributed) network environment. The machine 801 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 801 may include a hardware processor 802 (e.g., a central processing unit (CPU), a graphics processing unit (GPU), a hardware processor core, or any combination thereof), a main memory 803 and a static memory 804, some or all of which may communicate with each other via an interlink (e.g., bus) 805. The machine 801 may further include a display unit 806, an alphanumeric input device 807 (e.g., a keyboard), and a user interface (UI) navigation device 808 (e.g., a mouse). In an example, the display unit 806, alphanumeric input device 807 and ui navigation device 808 may be a touch screen display. The machine 801 may additionally include a storage device (e.g., drive unit) 809, a signal generation device 810 (e.g., a speaker), a network interface device 811, and one or more sensors 812, such as a global positioning system (GPS) sensor, compass, accelerometer, or other sensor. The machine 801 may include an output controller 816, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 809 may include a machine readable medium 813 that is non-transitory on which is stored one or more sets of data structures or instructions 814 (e.g., software) embodying or utilized by any one or more of the techniques or functions described herein. The instructions 814 may also reside, completely or at least partially, within the main memory 803, within static memory 804, or within the hardware processor 802 during execution thereof by the machine 801. In an example, one or any combination of the hardware processor 802, the main memory 803, the static memory 804, or the storage device 809 may constitute machine readable media.

While the machine readable medium 813 is illustrated as a single medium, the term “machine readable medium” may include a single medium or multiple media (e.g., a centralized or distributed database, or associated caches and servers) configured to store the one or more instructions 814.

The term “machine readable medium” may include any medium that is capable of storing, encoding, or carrying instructions for execution by the machine 801 and that cause the machine 801 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 814 may further be transmitted or received over a communications network 815 using a transmission medium via the network interface device 811 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 811 may include one or more physical jacks (e.g., Ethernet, coaxial, or phone jacks) or one or more antennas to connect to the communications network 815. In an example, the network interface device 811 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 801, and includes digital or analog communications signals or other intangible medium to facilitate communication of such software.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for electrochemical nutrient delivery in water, the method comprising:
deploying first and second electrodes within a water-based matrix; and
establishing a specified potential difference between the first and second electrodes, the first electrode defining an anode in an electrochemical cell, and the second electrode defining a cathode in the electrochemical cell, with the water-based matrix providing an electrolyte for the cell;
wherein:
at least one of the first and second electrodes comprises an inorganic nutrient; and
the establishing of the potential difference comprises eliciting delivery of the inorganic nutrient via controlled electrochemical nutrient delivery (CEND) in a water-based matrix, including the inorganic nutrient.

2. The method of claim 1, wherein the first electrode comprises a plurality of inorganic nutrients.

3. The method of claim 1, wherein the first electrode is clad with the inorganic nutrient.

4. The method of claim 3, wherein the inorganic nutrient comprises a metallic species.

5. The method of claim 4, wherein the inorganic nutrient includes alloy-forming metals and metalloids, including at least one of iron (Fe), Zinc (Zn), or Manganese (Mn).

6. The method of claim 4, wherein oxidation of the first electrode includes changing an oxidation state of the metallic species to elicit the electrochemical delivery.

7. The method of claim 6, wherein the metallic species comprises iron (Fe), and wherein the oxidation state is driven by the specified potential toward a state of Fe⁺² or Fe⁺³, or both.

8. The method of claim 1, comprising:
monitoring an indication of growth of a photoautotroph in the water-based matrix; and
in response to the monitoring:
controlling a delivery rate of the inorganic nutrient by modulating the specified potential difference; and
controlling cumulative nutrient delivery by establishing or adjusting a period of applied potential.

9. The method of claim 8, wherein the controlling the delivery of the inorganic nutrient includes reducing or eliminating the specified potential or driving the specified potential to an opposite polarity.

10. The method of claim 1, wherein:
the water-based matrix comprises an open or coastal marine environment including an aquatic reservoir, or other body of water; and
controlling delivery of inorganic nutrient via controlled electrochemical nutrient delivery (CEND) is based on a nutrient demand of a photoautotroph population of an ecosystem located in the aquatic reservoir or other body of water and to stimulate growth of or carbon capture via the photoautotroph population.

11. The method of claim 1 comprising wherein:
the water-based matrix is within a flow-controlled hydroponic system or a raceway pond; and
delivery of inorganic nutrient via controlled electrochemical nutrient delivery (CEND) is based on a specified target concentration of the inorganic nutrient in the water-based matrix and to stimulate growth of or carbon capture via a photoautotroph population in the water-based matrix.

12. The method of claim 11, comprising:
determining a concentration of the inorganic nutrient within the hydroponic system;
wherein the establishing the specified potential difference between the first and second electrodes or establishing a length of an electrical pulse for exciting the first or second electrode is based on the determined concentration of the inorganic nutrient and to alter the concentration toward the specified target concentration.

13. The method of claim 1, wherein controlling delivery of an inorganic nutrient via the CEND includes establishing a composition of a material of the first electrode for delivery of a specified amount or type of a target nutrient.

14. The method of claim 13, wherein the establishing the composition of the material includes an alloy, an intermetallic, or a metal-containing composite configured to deliver specified amount of the inorganic nutrient to be delivered from the first electrode during the CEND.

15. The method of claim 1, wherein the specified potential difference between the first and second electrodes is less than five volts (V) per cell pair.

16. The method of claim 1, comprising exciting the first and second electrodes, to establish the specified potential difference, via a source of renewable energy.

17. The method of claim 16, wherein the source of renewable energy includes electrical energy converted from kinetic or thermal energy of the water-based matrix, wind energy, or solar energy.

18. The method of claim 1, comprising exciting the first and second electrodes, to establish the specified potential difference, via a time-varying potential.

19. Them method of claim 18, comprising controlling the time-varying potential at a specified pulse repetition frequency (PRF) and a specified pulse width to define a program for eliciting delivery of the inorganic nutrient via the CEND.

20. A system comprising:
first and second electrodes configured for use in a marine environment, the first electrode defining an anode in an electrochemical cell, and the second electrode defining a cathode in the electrochemical cell, with the marine environment providing an electrolyte for the cell;
an excitation source electrically coupled with the first and second electrodes; and
a control circuit configured to establish a specified potential difference between the first and second electrodes using the excitation source;
wherein:
the first electrode comprises an inorganic nutrient; and
the control circuit is configured to establish the potential difference to elicit delivery of the inorganic nutrient via controlled electrochemical nutrient delivery (CEND) in the marine environment in a controlled manner using the specified potential difference.

21. The system of claim 20, wherein the anode comprises a linear electrode configuration.

22. The system of claim 20, wherein the cathode comprises a ring configuration.

23. The system of claim 20, wherein the anode comprises a rod or plate.

24. The system of claim 20, wherein anode is clad with the inorganic nutrient, comprising an electrochemically deliverable metallic species.

25. The system of claim 24, wherein the electrochemically deliverable metallic species includes at least one of iron (Fe), Zinc (Zn), or Manganese (Mn).

26. The system of claim 20, further comprising the marine environment.

27. The system of claim 26, wherein the marine environment is defined by a growth cell isolated from other growth cells.

28. The system of claim 27, wherein the marine environment includes a hydroponic reservoir.

29. The system of claim 20, wherein the excitation source is coupled to a renewable energy source.

30. The system of claim 29, wherein:
the renewable energy source is configured to supply energy to the excitation source derived from at least one of solar, wave, or wind energy.

31. The system of claim 30, wherein the excitation source is configured to establish the specified potential between the first and second electrodes using the renewable energy source without requiring a source of energy other than the renewable energy source.
